(19) United States
(12) Patent Application Publication
(10) Pub. No.: US 2019/0376919 A1
(43) Pub. Date: Dec. 12, 2019

(54) CONTACT COMBUSTION TYPE GAS SENSOR AND METHOD FOR MANUFACTURING THE SAME

(71) Applicant: YAMAHA FINE TECHNOLOGIES CO., LTD., Hamamatsu-shi (JP)

(72) Inventor: Atsuo HATTORI, Iwata-shi (JP)

(21) Appl. No.: 16/432,275

(22) Filed: Jun. 5, 2019

(30) Foreign Application Priority Data
Jun. 7, 2018 (JP) 2018-109675

(51) Int. Cl. G01N 27/16; G01N 33/00

(52) U.S. Cl. CPC G01N 27/16; G01N 33/005

(57) ABSTRACT

A contact combustion type gas sensor includes: a substrate; a catalytic layer that includes a catalyst for accelerating a chemical reaction of a detection target gas; a temperature measuring element that detects at least an increase in a temperature of the catalytic layer; and a support member that transfers heat generated by the chemical reaction toward the temperature measuring element and includes a support leg and a support body that are connected with each other, the support leg being provided between the substrate and the catalytic layer and separating the catalytic layer from the substrate, and the support body supporting the catalytic layer.

### CROSS-REFERENCE TO RELATED APPLICATION

Priority is claimed on Japanese Patent Application No. 2018-109675, filed Jun. 7, 2018, the content of which is incorporated herein by reference.

### BACKGROUND OF THE INVENTION

### Field of the Invention

The present invention relates to a contact combustion type gas sensor and a method for manufacturing the same.

### Description of Related Art

In general contact combustion type gas sensors, a catalytic layer and a heater are provided on a substrate. The catalytic layer acts as a catalyst for combustion of a combustible gas. The heater accelerates the combustion.

In contact combustion type gas sensors, a heat quantity of the combustion is measured by a temperature measuring element such as a thermopile, and thereby the quantity of the combustible gas is determined (e.g., see Japanese Unexamined Patent Application, First Publication No. 2001-99801 (hereinafter referred to as Patent Document 1)).

In the gas sensor disclosed in Patent Document 1, a catalytic layer is provided in a small area region on a substrate. This catalytic layer acts as a catalyst for combustion of a combustible gas. However, since the catalytic layer is restricted to being within a small area region, sufficient combustion heat from the combustible gas cannot be obtained. For this reason, it is impossible to increase the detection sensitivity with respect to a detection target gas.

### SUMMARY OF THE INVENTION

An object of the present invention is to provide a contact combustion type gas sensor capable of increasing the detection sensitivity with respect to a detection target gas and a method for manufacturing the same.

To solve the problem, according an aspect of the present invention, a contact combustion type gas sensor includes: a substrate; a catalytic layer that includes a catalyst for accelerating a chemical reaction of a detection target gas; a temperature measuring element that detects at least an increase in a temperature of the catalytic layer; and a support member that transfers heat generated by the chemical reaction toward the temperature measuring element and includes a support leg and a support body that are connected with each other, the support leg being provided between the substrate and the catalytic layer and separating the catalytic layer from the substrate, and the support body supporting the catalytic layer.

According to another aspect of the present invention, a method for manufacturing a contact combustion type gas sensor includes: providing a substrate; providing a catalytic layer that includes a catalyst for accelerating a chemical reaction of a detection target gas; providing a temperature measuring element that detects at least an increase in a temperature of the catalytic layer; and providing a support member that transfers heat generated by the chemical reaction toward the temperature measuring element and includes a support leg and a support body that are connected with each other, the support leg being provided between the substrate and the catalytic layer and separating the catalytic layer from the substrate, and the support body supporting the catalytic layer.

According to the contact combustion type gas sensor and the method for manufacturing a contact combustion type gas sensor, detection sensitivity with respect to a detection target gas can be enhanced.

### BRIEF DESCRIPTION OF THE DRAWINGS

FIG. 1A is a top view showing a contact combustion type gas sensor according to a first embodiment.

FIG. 1B is a sectional view taken along line B-B of FIG. 1A.

FIG. 2A is a top view sequentially showing a state in which the contact combustion type gas sensor is manufactured in a first manufacturing process.

FIG. 2B is a sectional view taken along line B-B of FIG. 2A.

FIG. 2C is a sectional view taken along line C-C of FIG. 2A.

FIG. 3A is a top view sequentially showing a state in which the contact combustion type gas sensor is manufactured in a second manufacturing process.

FIG. 3B is a sectional view taken along line B-B of FIG. 3A.

FIG. 3C is a sectional view taken along line C-C of FIG. 3A.

FIG. 4A is a top view sequentially showing a state in which the contact combustion type gas sensor is manufactured in a third manufacturing process.

FIG. 4B is a sectional view taken along line B-B of FIG. 4A.

FIG. 4C is a sectional view taken along line C-C of FIG. 4A.

FIG. 5A is a top view sequentially showing a state in which the contact combustion type gas sensor is manufactured in a fourth manufacturing process.

FIG. 5B is a sectional view taken along line B-B of FIG. 5A.

FIG. 5C is a sectional view taken along line C-C of FIG. 5A.

FIG. 6A is a top view showing a contact combustion type gas sensor according to a second embodiment.

FIG. 6B is a sectional view taken along line B-B of FIG. 6A.

FIG. 7A is a top view showing a contact combustion type gas sensor according to a third embodiment.

FIG. 7B is a sectional view taken along line B-B of FIG. 7A.

FIG. 8A is a top view sequentially showing the contact combustion type gas sensor in a first manufacturing process according to the third embodiment.

FIG. 8B is a sectional view taken along line B-B of FIG. 8A.

FIG. 8C is a sectional view taken along line C-C of FIG. 8A.

FIG. 9A is a top view sequentially showing the contact combustion type gas sensor in a second manufacturing process according to the third embodiment.

FIG. 9B is a sectional view taken along line B-B of FIG. 9A.

FIG. 9C is a sectional view taken along line C-C of FIG. 9A.

FIG. 10A is a top view showing a contact combustion type gas sensor according to a fourth embodiment.

FIG. 10B is a sectional view taken along line B-B of FIG. 10A.

FIG. 11A is a top view showing a contact combustion type gas sensor according to a fifth embodiment.

FIG. 11B is a sectional view taken along line B-B of FIG. 11A.

### DETAILED DESCRIPTION OF THE INVENTION

Hereinafter, a contact combustion type gas sensor and a method for manufacturing the same to which embodiments of the present invention is applied will be described. In first to fifth embodiments and their modifications, a case where a detection target gas is hydrogen will be described by way of example. The detection target gas may be a combustible gas other than hydrogen. Further, in each embodiment, elements, members, etc. which are the same as one another are denoted by the same reference signs, and description thereof is sometimes omitted or simplified. In the description of the embodiments of the present invention, when viewed from a substrate (to be described below), a direction in which a catalytic layer is provided is defined as upward, and a direction opposite to this direction is defined as downward. Further, with regard to layer-shaped, film-shaped, or plate-shaped elements, members, or the like, an upper surface is defined as a top (a surface), and a lower surface is defined as a bottom.

### First Embodiment

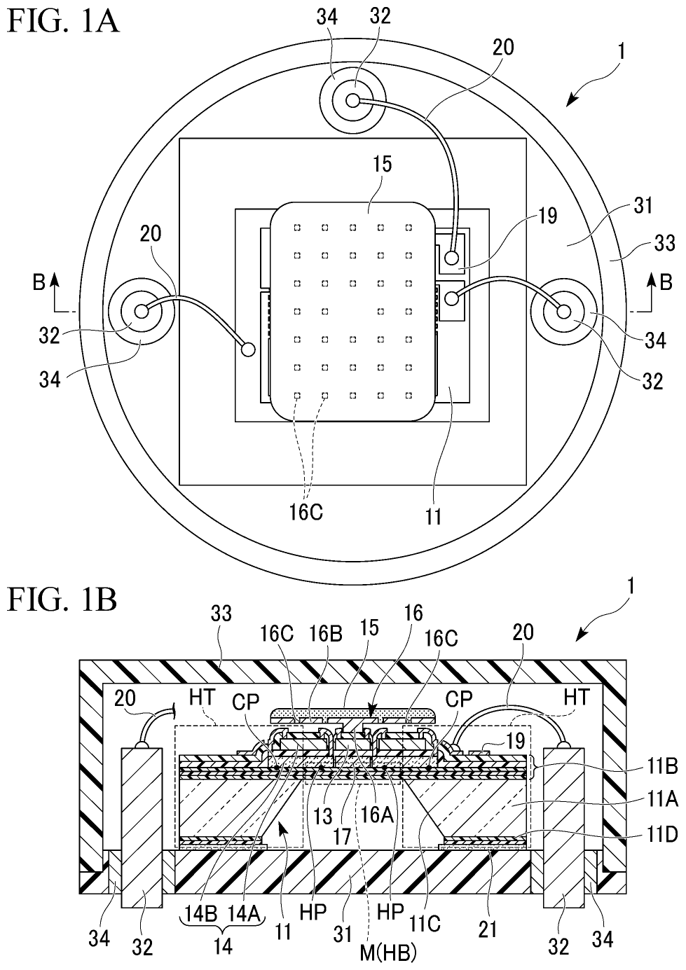

A first embodiment of the present invention will be described. FIG. 1A is a top view showing a contact combustion type gas sensor according to a first embodiment. FIG. 1B is a sectional view taken along line B-B of FIG. 1A. As shown in FIGS. 1A and 1B, a contact combustion type gas sensor 1 according to the present embodiment includes a substrate 11. The substrate 11 includes a substrate body 11A, an upper insulating layer 11B, and a lower insulating layer 11D. The substrate body 11A is formed of a material having excellent thermal conductivity. For example, the substrate body 11A may be formed of single-crystal silicon, but may be formed of another material. The upper insulating layer 11B is made up of a plurality of insulating films provided on a top side of the substrate body 11A. The upper insulating layer 11B is formed of a material having an insulation property and low thermal conductivity, for example a silicon oxide film or a silicon nitride film. The lower insulating layer 11D is formed on the bottom of the substrate body 11A in a layered shape. A recess 11C is formed on a bottom side of the substrate 11. The recess 11C is formed by etching a part of the substrate body 11A (an approximately central portion shown in FIG. 1B) and a part of the lower insulating layer 11D (an approximately central portion shown in FIG. 1B). A portion of the upper insulating layer 11B in the substrate 11, which is a portion (an approximately central portion shown in FIG. 1B) exposed from the recess 11C after the recess 11C and the lower insulating layer 11D are etched, is a membrane M or a heat-insulated part HB. Further, other portions of the substrate 11, namely, a portion of the upper insulating layer 11B, which is a portion (an unexposed portion) other than the portion exposed from the recess 11C after the recess 11C and the lower insulating layer 11D are etched, and the substrate body 11A are a non-heat-insulated part HT. The non-heat-insulated part HT includes the substrate body 11A having an excellent thermal conductivity and a base material 31 that is thermally connected to the substrate body 11A via a die bonding material 21 having an excellent thermal conductivity. Moreover, the non-heat-insulated part HT faces a gas flowing into the contact combustion type gas sensor 1 over a wide area. Therefore, the non-heat-insulated part HT is influenced by a temperature of the gas, and its temperature becomes equal or close to the temperature of the gas flowing into the contact combustion type gas sensor 1. In contrast, since the heat-insulated part HB has a low thermal conductivity and is not thermally connected to the non-heat-insulated part HT, the heat-insulated part itself and members provided in the heat-insulated part are thus hardly influenced by the temperature of the gas flowing into the contact combustion type gas sensor 1.

The die bonding material 21 is provided on a lower surface of the substrate 11, and the base material 31 is provided under the die bonding material 21. The substrate 11 is bonded and fixed to the base material 31 by the die bonding material 21.

As shown in FIG. 1A, three electrodes 32 are provided on portions of the base material 31 at which the substrate 11 is not provided. As shown in FIG. 1B, the electrodes 32 are provided through the base material 31. An insulating material 34 is filled between the base material 31 and each electrode 32. Insulation between the base material 31 and each electrode 32 is maintained by the insulating material 34. That is, the insulating material 34 electrically insulates each electrode 32 and the base material 31 from each other. A filter 33 is provided on the base material 31. However, in FIG. 1A, a portion of the filter 33 which covers an upper side of the base material 31 is not shown. The filter 33 covers an upper portion of the contact combustion type gas sensor 1. For this reason, the filter 33 also covers the substrate 11, a heater 13, a temperature measuring element 14, and so on which are provided in the contact combustion type gas sensor 1. The filter 33 has gas permeability, and removes dust included in the gas flowing into the contact combustion type gas sensor 1. The filter 33 is formed of, for example, a sintered metal made from stainless steel or brass, a stainless steel wire net, or a porous ceramic.

A heat transfer film 17, the heater 13, and the temperature measuring element 14 are provided between and on a plurality of insulating layers that constitute the upper insulating layer 11B. The heat transfer film 17 has excellent thermal conductivity and electrical conductivity, and is formed of, for example, polycrystalline silicon. The heat transfer film 17 transfers heat supplied partly from the heater 13 and the other members, and equalizes its own temperature distribution. The heat transfer film 17 may be omitted. The heater 13 and the temperature measuring element 14 are formed of, for example, polycrystalline silicon. The temperature of the heater 13 is raised by supplying a driving current from an external power supply (not shown). The temperature measuring element 14 is formed of a thermopile. The thermopile is formed of a plurality of thermocouples that are connected in series. Each thermocouple includes an upper temperature measuring element 14A formed of an N-type semiconductor and a lower temperature measuring element 14B formed of a P-type semiconductor. A hot junction HP of each thermocouple is provided in the membrane M, namely the heat-insulated part HP. Further, a cold junction CP of each thermocouple is provided in the non-heat-insulated part HT.

A support member 16 is provided above the heater 13 and on the upper insulating layer 11B, and a catalytic layer 15 is provided above and on the support member 16.

The support member 16 is formed of a high thermoconductive material. For example, the support member 16 may be formed of a metal such as aluminum, but it may be formed of another metal or another material. The support member 16 thermally connects the heater 13 and the temperature measuring element 14 to the catalytic layer 15. For this reason, the heater 13 becomes a heating means for heating the catalytic layer 15. The support member 16 has an approximate T shape when viewed from the side.

The support member 16 is made up of a support leg 16A and a support body 16B. The support leg 16A has, for example, a prismatic shape, and stands upward on the upper insulating layer 11B and is almost perpendicular to the substrate body 11A. As shown in FIG. 1A, the support body 16B has, for example, a rectangular shape in which the corners are chamfered. An area of the support body 16B when viewed from above is larger than that of the membrane M. A plurality of through-holes 16C are formed in the support body 16B, and are aligned in a matrix. The through-holes 16C of the support body 16B may be concave hole parts in which a surface of the support body 16B is recessed without passing through the support body 16B.

The catalytic layer 15 is supported by the support body 16B. The catalytic layer 15 enters the through-holes 16C. The catalytic layer 15 has a greater thickness than the support body 16B in the support member 16. Here, the thickness of the catalytic layer 15 is a thickness of a portion excluding a portion of the catalytic layer 15 which enters the through-holes 16C. The catalytic layer 15 functions as a catalyst that allows a chemical reaction between oxygen in air and hydrogen to be detected. Heat generated in the catalytic layer 15 is efficiently collected by the support body 16B formed of a high thermoconductive material, and is transferred to the hot junctions HP of the temperature measuring element 14 via the support leg 16A.

Surface wirings 19 are provided on a top layer of the upper insulating layer 11B. The surface wirings 19 are electrically connected to the electrodes 32 via wires 20. The surface wirings 19 are connected to the heater 13 and the temperature measuring element 14. A driving current is supplied to the heater 13 via the electrodes 32, the wires 20, and the surface wirings 19. The heater 13 is raised in temperature by a driving current supplied from an external control device (not shown), and heats the catalytic layer 15. The hot junctions HP of the temperature measuring element 14 are provided at positions at which they are thermally connected to the catalytic layer 15 in the substrate 11. The temperature measuring element 14 outputs a voltage generated by a temperature differential between the hot junction HP and the cold junction CP to the control device as an output voltage. The control device measures an increase in temperature of the catalytic layer 15 relative to the set temperature of the heater 13 on the basis of the voltage output from the temperature measuring element, and detects a concentration of hydrogen (an amount of gas).

A process of detecting hydrogen using the contact combustion type gas sensor 1 according to the present embodiment will be described. First, a test target (e.g., an airtight container) that is a target with respect to inspecting for leakage of hydrogen is installed in a chamber, and the contact combustion type gas sensor 1 is installed in the test target. Next, hydrogen is filled between the chamber and the test target, and a temperature of the heater 13 is raised by the control device. A voltage output from the temperature measuring element 14 is measured to detect a concentration of the hydrogen.

When hydrogen is present in the test target in the process of detecting hydrogen, the hydrogen reacts with oxygen in air around the catalytic layer 15, so that water is generated. Due to reaction heat in this case, a temperature of the catalytic layer 15 rises. When the temperature of the catalytic layer 15 rises, the temperature of the hot junctions HP in the heat-insulated part HB (the membrane M) of the substrate 11 connected thermally to the catalytic layer 15 rises. On the other hand, since the non-heat-insulated part HT of the substrate 11 (the portion other than the membrane M of the substrate 11) is not thermally connected to the catalytic layer 15, the temperature of the catalytic layer 15 remains almost constant even if its temperature rises.

The hot junctions HP are provided in the heat-insulated part HB (the membrane M), the cold junctions CP are provided in the non-heat-insulated part HT of the substrate 11 (the portion other than the membrane M of the substrate 11), and the temperature measuring element 14 outputs a voltage generated by a temperature differential between the hot junction HP and the cold junction CP to the control device. When the temperature of the catalytic layer 15 rises due to the reaction between hydrogen and oxygen, the temperature differential between the hot junction HP and the cold junction CP in the temperature measuring element 14 increases, and the voltage output to the control device also increases, compared to the case where the temperature of the catalytic layer 15 remains constant. The control device detects a concentration of hydrogen in the test target on the basis of the output voltage. The concentration of hydrogen in the test target is detected in the control device on the basis of the voltage output from the temperature measuring element 14 which is compared with a voltage output from the temperature measuring element 14 when the temperature of the catalytic layer 15 is not raised.

In the contact combustion type gas sensor 1 of the present embodiment described above, the temperature of the catalytic layer 15 is measured by the temperature measuring element 14, and thus the concentration of hydrogen is detected. Here, the catalytic layer 15 is provided on the support member 16, and is supported by the support body 16B. The support leg 16A almost vertically stands upward. The support body 16B is supported by the support leg 16A in a state in which the support leg 16A is thermally connected to the hot junctions HP of the temperature measuring element 14. Further, since the support body 16B is thermally connected to the heater 13, heat of the heater 13 can be supplied to the catalytic layer 15. Therefore, the temperature of the catalytic layer 15 can be suitably raised. Further, since the support body 16B is provided at a position apart from the substrate body 11A, an area thereof can be increased. By increasing the area of the support body 16B, an area of the catalytic layer 15 which enables contact with hydrogen and oxygen can also increased. When the area of the catalytic layer 15 which enables contact with hydrogen and oxygen is increased, the amount of reaction heat of hydrogen and oxygen can thereby be further increased. Thus, the detection sensitivity with respect to a detection target gas can be enhanced.

Further, the temperature of the catalytic layer 15 is raised by the heater 13. For this reason, a reaction of hydrogen and oxygen caused by the catalytic layer 15 can be accelerated to become more active. Further, when the temperature of the heater 13 is set to a temperature equal to or higher than the boiling point of water, for example, 120° C., water generated on a surface of the catalytic layer 15 is evaporated, and thereby a drop in reaction rate caused by a water film of the surface of the catalytic layer 15 can be prevented. Therefore, the detection sensitivity with respect to the detection target gas can be further enhanced. Further, the temperature measuring element 14 is formed of a thermopile. For this reason, a plurality of N-type semiconductor elements and a plurality of P-type semiconductor elements are connected in series, and thereby an output voltage can be increased to detect a temperature, so that detection accuracy can be enhanced.

Further, the support leg 16A and the support body 16B are formed of a high thermoconductive material. For this reason, the heat from the heater 13 can be efficiently transferred to the catalytic layer 15, and the heat generated at the catalytic layer 15 can be more efficiently collected and transferred to the hot junctions HP of the temperature measuring element 14 via the support leg 16A. Therefore, since the reaction heat of hydrogen and oxygen can be collected more efficiently, the detection sensitivity with respect to the detection target gas can be enhanced.

Further, the thickness of the support body 16B is smaller than that of the catalytic layer 15. For this reason, the area of the catalytic layer 15 which enables contact with hydrogen and oxygen is increased, and thereby even if the catalytic layer 15 is enlarged, a rise in heat capacity of the support body 16B can be minimized. Therefore, deterioration in responsiveness of the contact combustion type gas sensor 1 can be suppressed.

Further, the plurality of through-holes 16C are provided in the support body 16B. For this reason, the catalytic layer 15 can be provided by advancing the catalytic layer 15 into the through-holes 16C. Thus, close contact between the catalytic layer 15 and the support body 16B is improved, and the catalytic layer 15 can be supported by the support body 16B in a stable state. The number of through-holes 16C may not be a large number but a small number, or a single number (one). Thus, there may be one or more through-holes 16C.

Further, when viewed in a laminating direction, the support body 16B has a larger area than the membrane M provided on the substrate body 11A. For this reason, the area of the support body 16B and therefore the area of the catalytic layer 15 which enables contact with hydrogen and oxygen can be further increased. Therefore, since the reaction heat of hydrogen and oxygen can be further increased, the detection sensitivity with respect to the detection target gas can be enhanced.

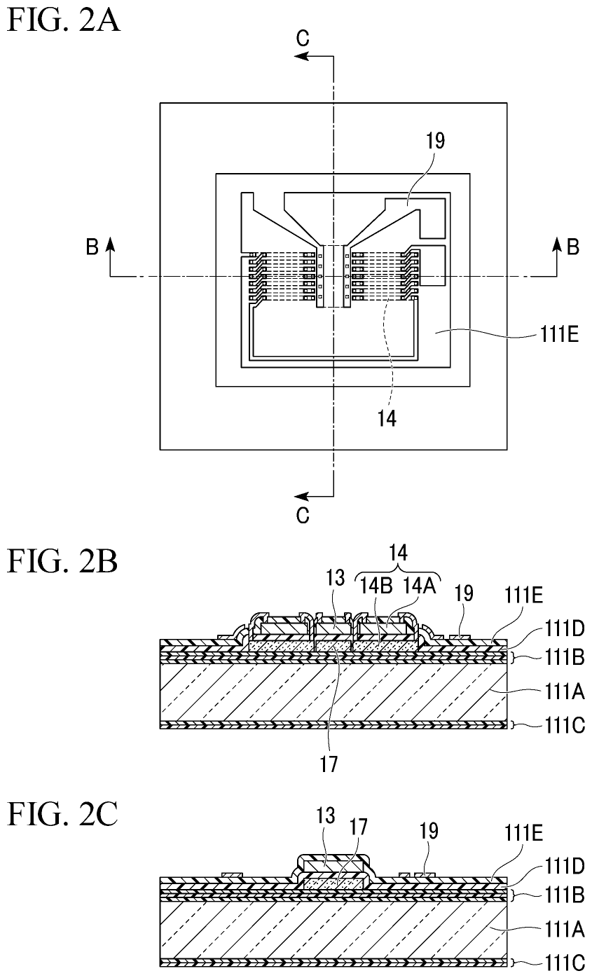

Next, a method for manufacturing the contact combustion type gas sensor 1 according to the first embodiment will be described. FIGS. 2A to 5C are views sequentially showing states in which the contact combustion type gas sensor is manufactured in processes of manufacturing the contact combustion type gas sensor. In FIGS. 2A to 5C, FIGS. 2A, 3A, 4A, and 5A are top views. FIGS. 2B, 3B, 4B, and 5B are sectional views taken along lines B-B of FIGS. 2A, 3A, 4A, and 5A. FIGS. 2C, 3C, 4C, and 5C are sectional views taken along lines C-C of FIGS. 2A, 3A, 4A, and 5A. As shown in FIG. 2, when the contact combustion type gas sensor 1 is manufactured, a three-layered insulation film layer 111B is first provided on a single-crystal silicon layer 111A serving as the substrate body 11A after being manufactured, and a two-layered insulation film layer 111C is provided under the single-crystal silicon layer 111A. The insulation film layers 111B and 111C are formed using a suitable method such as a plasma CVD method or a LP-CVD method. Other insulation film layers can also be formed by the same method. The two-layered insulation film layer 111C serves as the lower insulating layer 11D shown in FIG. 1B. For example, the three-layered insulation film layer 111B has upper and lower layers formed of a silicon oxide film (SiO₂), and an intermediate layer formed of a silicon nitride film (Si₃N₄). Further, the two-layered insulation film layer 111C has an upper layer formed of a silicon oxide film (SiO₂), and a lower layer formed of a silicon nitride film (Si₃N₄).

Next, a semiconductor film serving as a heat transfer film 17 and a lower temperature measuring element 14B is formed. The semiconductor film is formed, for example, by forming polycrystalline silicon using a pressure reduction CVD method and introducing impurities such as phosphor using an ion implantation method. After the ion implantation, lamp annealing may be performed to activate ions. Further, instead of ion implantation, impurities such as phosphor may be thermally diffused by a diffusion furnace. Further, instead of polycrystalline silicon, iron silicide, SiGe, bismuth antimony, or the like may be formed into a film by a sputtering method. Other semiconductor films can also be formed by the same methods.

The semiconductor film formed in this way is patterned into a desired pattern shape, for example, using a photolithography technique, and is etched to form the heat transfer film 17 and the lower temperature measuring element 14B. An insulation film layer 111D is formed on the heat transfer film 17, the lower temperature measuring element 14B, and the upper layer of the insulation film layer 111B. Then, a heater 13 and an upper temperature measuring element 14A are formed above the heat transfer film 17 and the lower temperature measuring element 14B with the insulation film layer 111D therebetween, and an insulation film layer 111E is formed on the insulation film layer 111D. The insulation film layers 111B, 111D and 111E form the upper insulating layer 11B shown in FIG. 1B. For example, the insulation film layer 111D is formed of a silicon oxide film (SiO₂), and the insulation film layer 111E is formed of a silicon nitride film (Si₃N₄).

Next, a contact hole pattern having a desired shape is formed on the insulation film layer 111E, for example, using a photolithography technique, thereby partly exposing the heater 13 and the temperature measuring element 14. Afterward, the insulation film layer 111E is etched. For example, a conductive film may be formed of platinum, for example, using a sputtering method, and subjected to etching or the like, thereby forming surface wirings 19.

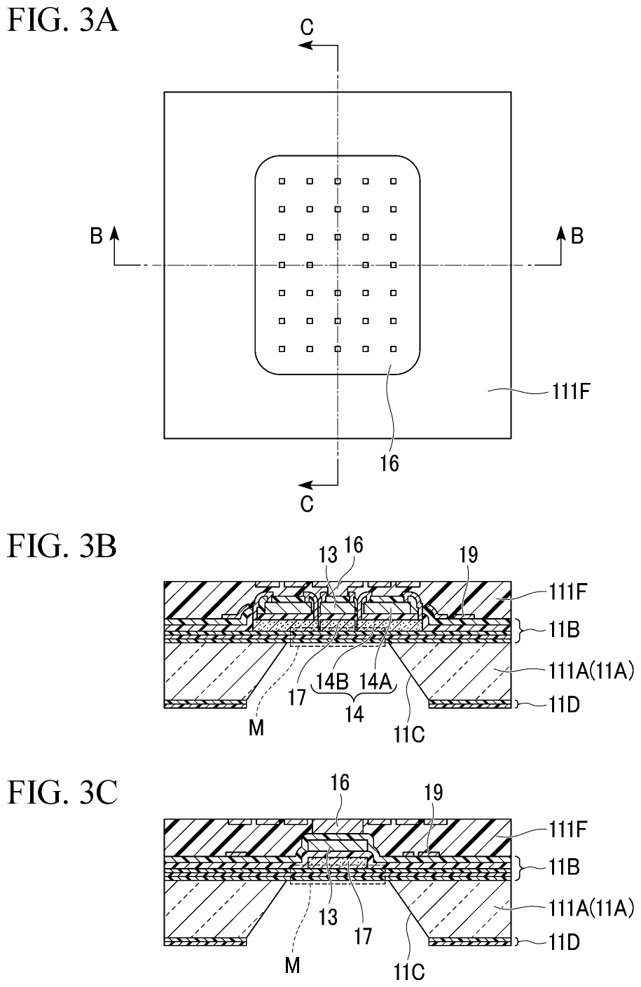

Then, as shown in FIGS. 3A to 3C, an insulation film layer (a sacrificial film layer) 111F is further formed on the upper insulating layer 11B and the surface wirings 19, and a support member recess for forming the support member 16 shown in FIG. 1B is formed thereon using a photolithography technique. For example, the insulation film layer 111F is formed of SiO₂. Next, a conductive film is formed on the top of the insulation film layer 111F by a plating method. The conductive film is formed of, for example, copper. Further, the conductive film remains in the support member recess in a CMP technique, thereby forming a support member 16. Since the support member 16 is adjacent to the heater 13 with the single-layered insulation film layer 111E therebetween, the heater 13 and the support member 16 are thermally connected, and heat of the heater 13 is transferred to the support member 16, so that a temperature of the support member 16 is approximately the same as that of the heater 13. Afterward, the single-crystal silicon layer 111A is etched from a lower surface (the bottom) thereof, and a recess 11C is formed. By forming the recess 11C, a part of the upper insulating layer 11B (an approximately central portion shown in FIG. 1B) serves as a membrane M, which becomes a substrate body 11A.

Next, the insulation film layer 111F is etched. Here, for example, etching using buffered hydrofluoric acid is performed. The insulation film layer 111F, and the insulation film layer 111E and the surface wirings 19 under the insulation film layer 111F are respectively formed of a silicon oxide film (SiO₂), a silicon nitride film (Si₃N₄), and platinum (Pt). The insulation film layers 111F and 111E greatly differ in etching rate for the buffered hydrofluoric acid. For this reason, when the insulation film layer 111F is etched, the insulation film layer 111E under the insulation film layer 111F functions as an etching stopper. A number of through-holes 16C aligned in a matrix are provided in the support body 16B. Since the buffered hydrofluoric acid can enter from the through-holes 16C, an etching time of the insulation film layer 111F is shortened.

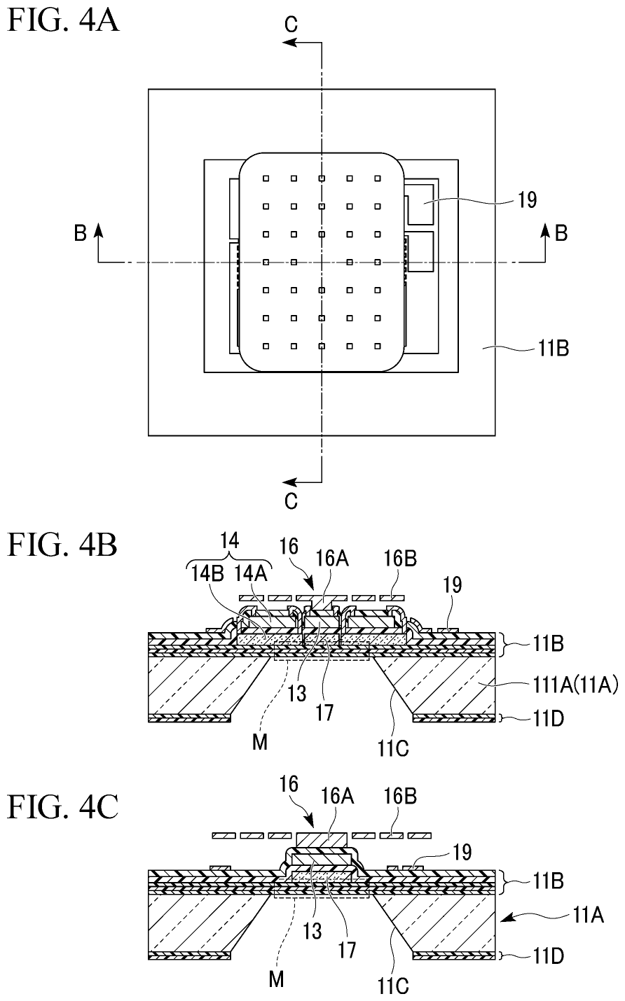

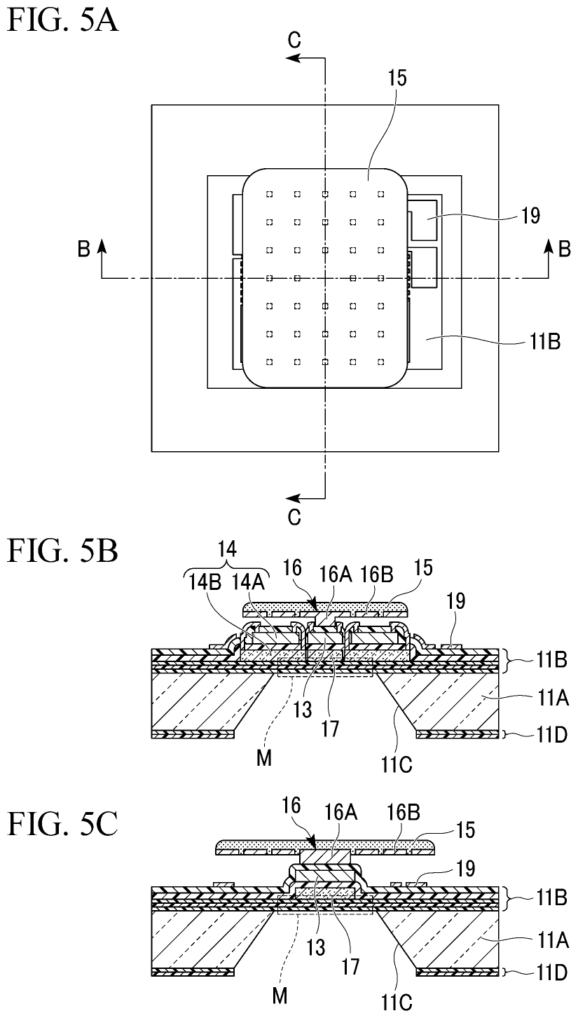

When the insulation film layer 111F is etched, the support member 16 and the surface wirings 19 are exposed as shown in FIGS. 4A to 4C. Afterward, a catalytic paste is applied to and burned on the top of the support body 16B at the support member 16. For example, the catalytic paste is made by mixing porous alumina powder and organic vehicles (α-terpineol and di-n-butyl phthalate) on which catalytic fine particles formed of platinum are supported. The catalytic paste is applied to a surface of the support body 16B of the support member 16 using a dispenser, and then is burned in an oven, for example, under conditions of 700° C. and one hour. Thus, as shown in FIGS. 5A to 5C, the catalytic layer 15 can be formed on the support body 16B of the support member 16. The catalytic layer 15 is burned before the insulation film layer 111F is etched, and may be burned after or before the recess 11C is formed on the bottom of the substrate body 11A.

Afterward, a lower surface of the lower insulating layer 11D is attached and fixed to a base material 31 with a die bonding material 21, and the surface wirings 19 are connected to electrodes 32 via wires 20. The substrate 11 and the heater 13, the temperature measuring element 14, and the catalytic layer 15 that are mounted on the substrate 11 are covered with a filter 33. Thus, the contact combustion type gas sensor 1 is completed.

In this way, when the contact combustion type gas sensor 1 is manufactured, the insulation film (the sacrificial film) is provided and then the support member 16 and the catalytic layer 15 that is supported by and the support body 16B are provided. For this reason, the support member 16 and the catalytic layer 15 can be formed with ease and accuracy. The support member 16 is provided by laminating copper or the like on the substrate 11, and thereby the support member 16 and the catalytic layer 15 can be formed by a flow of the process of forming the substrate 11. Therefore, since the contact combustion type gas sensor 1 can be manufactured in a manufacturing process using high-precision microfabrication equipment installed in a clean room, mass production can be performed with a high quality and a high yield at a low cost.

### Second Embodiment

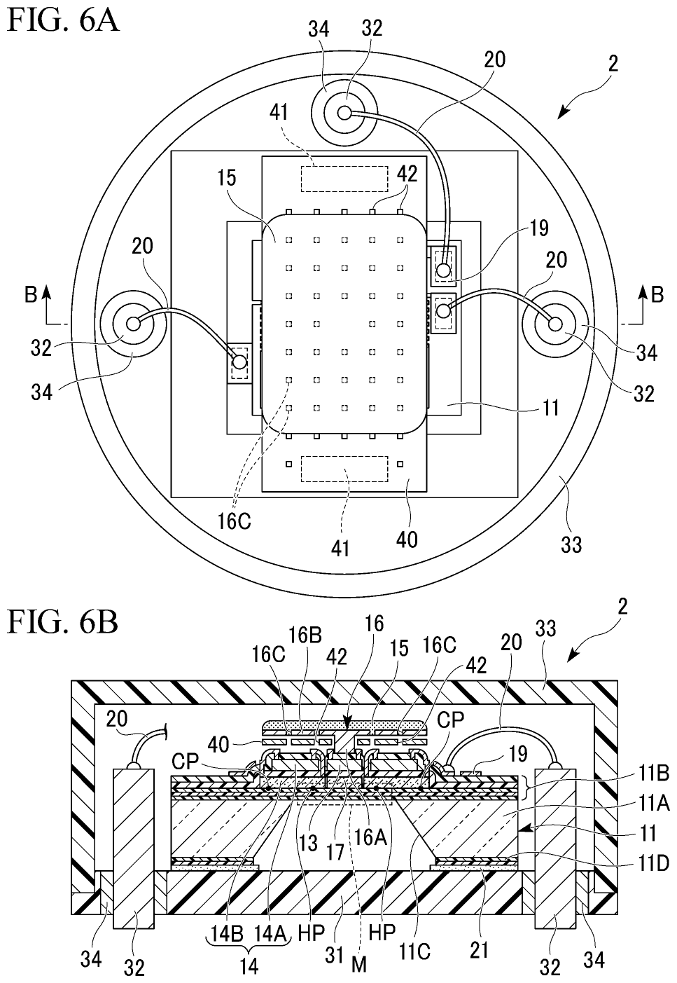

Next, a second embodiment of the present invention will be described. FIG. 6A is a top view showing a contact combustion type gas sensor according to a second embodiment. FIG. 6B is a sectional view taken along line B-B of FIG. 6A. As shown in FIGS. 6A and 6B, the contact combustion type gas sensor 2 according to the present embodiment is mainly different from that of the first embodiment in that a thermal barrier 40 is provided between a substrate 11 and a support body 16B.

The thermal barrier 40 is supported by thermal barrier support legs 41 that stand upward to be almost perpendicular to the substrate 11. As shown in FIG. 6A, the thermal barrier 40 has a thin plate shape, and a thickness thereof is approximately the same as that of the support body 16B. Further, the thermal barrier 40 and the thermal barrier support legs 41 are integrally formed.

A plurality of through-holes 42 are provided in the thermal barrier 40. Like a plurality of through-holes 16C, the plurality of through-holes 42 are formed in a state in which they are aligned in a matrix. The through-holes 42 provided in the thermal barrier 40 in a region where the thermal barrier 40 overlaps the support body 16B when viewed from above are formed such that positions thereof are coincident with those of the through-holes 16C provided in the support body 16B. The positions of the through-holes 42 may not necessarily coincident with those of the through-holes 16C.

A part of each thermal barrier support leg 41 is thermally and electrically connected to the substrate 11. Further, both the thermal barrier 40 and the thermal barrier support legs 41 are formed of a material that is excellent in thermal conductivity and electric conductivity, for example a metal such as copper. One ends of the wires 20 are connected to the electrodes 32, and the other ends of the wires 20 are electrically connected to the surface wirings 19. The heater 13 is supplied with a driving current from a control device (not shown) via the wires 20.

The contact combustion type gas sensor 2 according to the present embodiment described above operates in the same manner as the contact combustion type gas sensor 1 according to the first embodiment. Further, the contact combustion type gas sensor 2 according to the present embodiment includes the thermal barrier 40, and the thermal barrier 40 is interposed between the catalytic layer 15 and the temperature measuring element 14 and between the support body 16B and the temperature measuring element 14. For this reason, the thermal barrier 40 can block heat discharged from the catalytic layer 15 and the support body 16B, and inhibit direct movement of heat from the catalytic layer 15 to the temperature measuring element 14, especially movement of heat caused by radiant heat or convection. Therefore, the temperature of the catalytic layer 15 based on the difference in temperature between cold and hot junctions CP and HP can be suitably detected by the temperature measuring element 14. Further, the thermal barrier 40 can function as a shield of the temperature measuring element 14. That is, the thermal barrier 40 electrically shields the temperature measuring element 14. Accordingly, noise is reduced. Therefore, detection accuracy of the temperature of the catalytic layer 15, and what is more, detection sensitivity with respect to a detection target gas can be further enhanced.

Further, the thermal barrier 40 is provided in the substrate 11 via the thermal barrier support legs 41 having an excellent thermal conductivity. For this reason, the temperature of the thermal barrier 40 can further approximate that of the substrate 11.

Therefore, the movement of heat from the catalytic layer 15 to the substrate 11 due to the thermal barrier 40 can be blocked more adequately. As a result, the detection accuracy of the temperature of the catalytic layer 15, and what is more, the detection sensitivity with respect to the detection target gas can be further enhanced.

### Third Embodiment

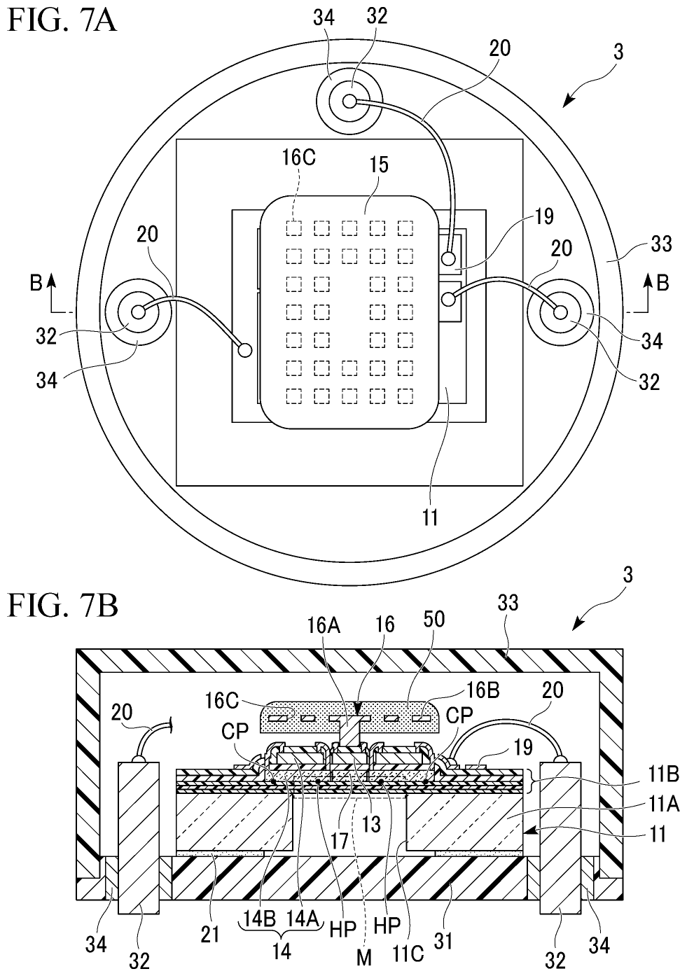

Next, a third embodiment will be described. FIG. 7A is a top view showing a contact combustion type gas sensor according to a third embodiment. FIG. 7B is a sectional view taken along line B-B of FIG. 7A. As shown in FIGS. 7A and 7B, the contact combustion type gas sensor 3 according to the present embodiment is mainly different from that of the first embodiment in that a catalytic layer 50 is provided on both an upper surface and a lower surface (the top and the bottom) of a support body 16B.

As shown in FIG. 7B, the catalytic layer 50 is provided on the upper and lower surfaces of the support body 16B. The catalytic layer 50 provided on the upper surface of the catalytic layer 50 has a shape in which upper sides of a cross section having an approximate quadrilateral shape are chamfered. Further, a portion of the catalytic layer 50 which is provided on an upper side of the support body 16B has approximately the same thickness as a portion of the catalytic layer 50 which is provided on a lower side of the support body 16B. The catalytic layer 50 protrudes outward beyond an outer circumference of the support body 16B, and is formed to completely surround the support body 16B. On the lower surface of the support body 16B, the catalytic layer 50 is provided to surround a portion where the support leg 16A and the support body 16B are joined. Further, the catalytic layer 50 passes through the through-holes 16C provided in the support body 16B, and joins the upper and lower surfaces of the support body 16B.

Further, an aspect of the substrate 11 in the present embodiment is slightly different from that in the first embodiment. To be specific, a cross section of a recess 11C provided in a substrate body 11A has an approximately rectangular shape in the present embodiment, but it has an approximately trapezoidal shape in the first embodiment. Further, no lower insulating layer is provided at a lower side of the substrate body 11A, and a lower surface of the substrate body 11A is directly attached and fixed to a base material 31 by a die bonding material 21.

The contact combustion type gas sensor 3 according to the present embodiment described above operates in the same manner as the contact combustion type gas sensor 1 according to the first embodiment. Further, the contact combustion type gas sensor 3 according to the present embodiment includes the catalytic layer 50 on both the upper surface (the top) and the lower surface (the bottom) of the support body 16B in the support member 16. For this reason, an area of the catalytic layer 50 which enables contact with hydrogen and oxygen can be further increased. Therefore, since reaction heat of hydrogen and oxygen can be further increased, detection sensitivity with respect to a detection target gas can be enhanced. Further, since the catalytic layer 50 is integrally formed to enclose the support body 16B, defects such as peeling of the catalytic layer 50 from the support body 16B can be remarkably reduced.

Next, a method for manufacturing the contact combustion type gas sensor 3 according to the present embodiment will be described, especially centering on a characteristic catalyst producing portion. When the contact combustion type gas sensor 3 is manufactured, the processes up to the portion shown in FIG. 2C are common with the processes of manufacturing the contact combustion type gas sensor 1 in the first embodiment.

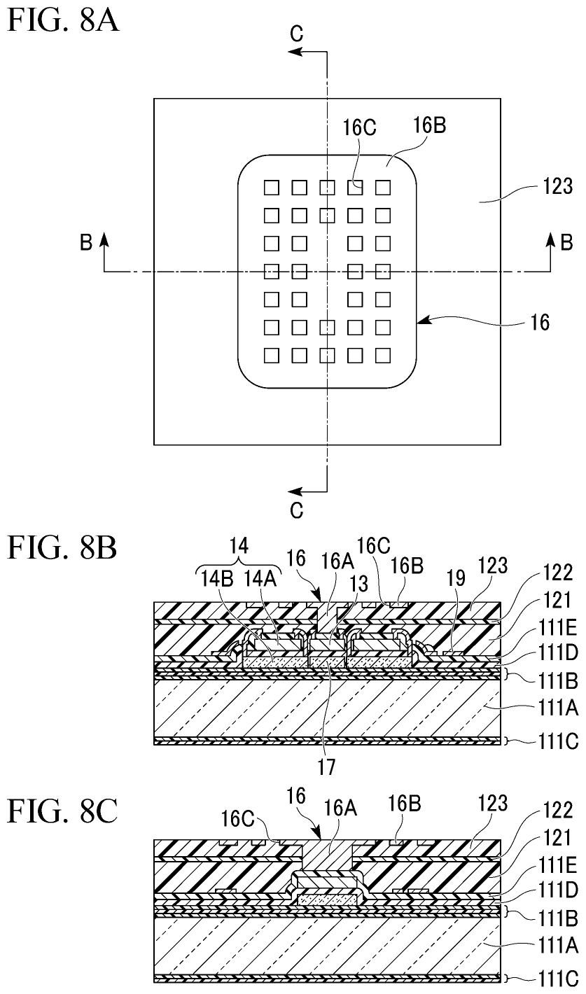

Next, in the first embodiment, the insulation film layer (the sacrificial film layer) 111F shown in FIGS. 3A to 3C is formed in one layer. However, in the present embodiment, as shown in FIGS. 8A to 8C, a first sacrificial film layer 121, a second sacrificial film layer 122, and a third sacrificial film layer 123 are provided. The first sacrificial film layer 121 and third sacrificial film layer 123 are formed of, for example, a silicon oxide film (SiO₂), and the second sacrificial film layer 122 is formed of, for example, a silicon nitride film (Si₃N₄). The third sacrificial film layer 123 is set to the same thickness as the catalytic layer 50 provided on the lower side of the support body 16B. Then, the first sacrificial film layer 121, the second sacrificial film layer 122, and the third sacrificial film layer 123 are subjected to etching or the like just like the insulation film layer 111F subjected to etching or the like in the first embodiment, and thus a support member 16 having a shape along a recess formed by etching is provided.

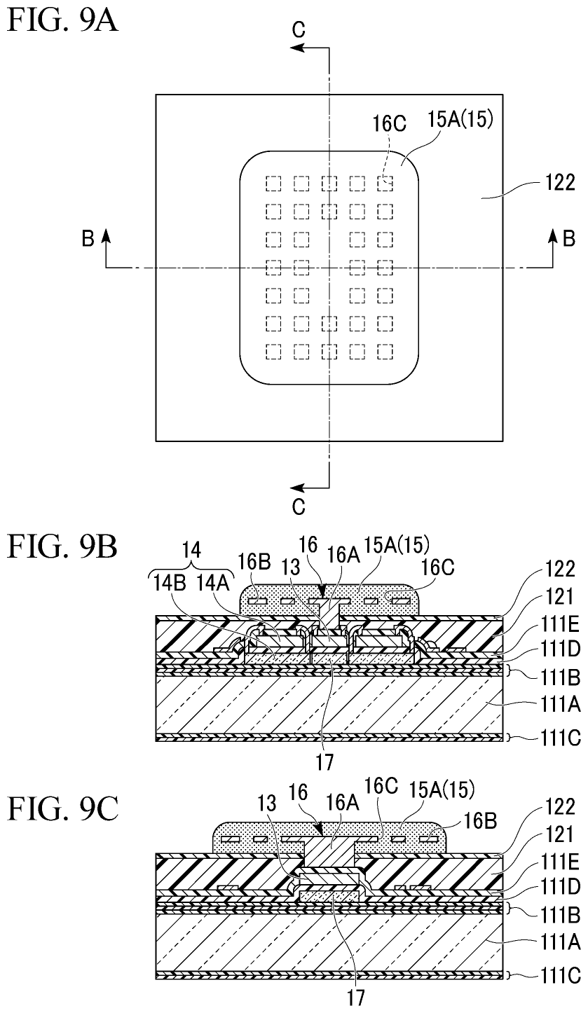

Afterward, the third sacrificial film layer 123 is removed. As shown in FIGS. 9A to 9C, a catalytic paste 15A is applied to and burned on an upper surface (the top) and a lower surface (the bottom) of a support body 16B, and a catalytic layer 50 is provided. When the catalytic paste 15A is applied, an application thickness, area, density, and viscosity of the catalytic paste, and what is more, a size, number, and arrangement of the through-holes 16C are adjusted such that the catalytic paste applied to the support body 16B reaches a surface of the second sacrificial film layer 122. Thus, the catalytic layer 50 is formed along the surface of the second sacrificial film layer 122. Afterward, the catalytic layer 50 is burned in an oven, and thereby the catalytic layer 50 is provided. Afterward, the second sacrificial film layer 122 and the first sacrificial film layer 121 are removed at an appropriate time. The catalytic paste may be applied and burned a plurality of times.

In this way, in a case where the contact combustion type gas sensor 3 according to the third embodiment is manufactured, or the catalytic layer 50 is formed on the lower surface (the bottom) of the support body 16B, the sacrificial film layer (the third sacrificial film layer 123) corresponding to a thickness of the catalytic layer 50 is formed. For this reason, the thickness of the catalytic layer 50 when the catalytic layer 50 is formed on the lower surface of the support body 16B can be easily adjusted.

### Fourth Embodiment

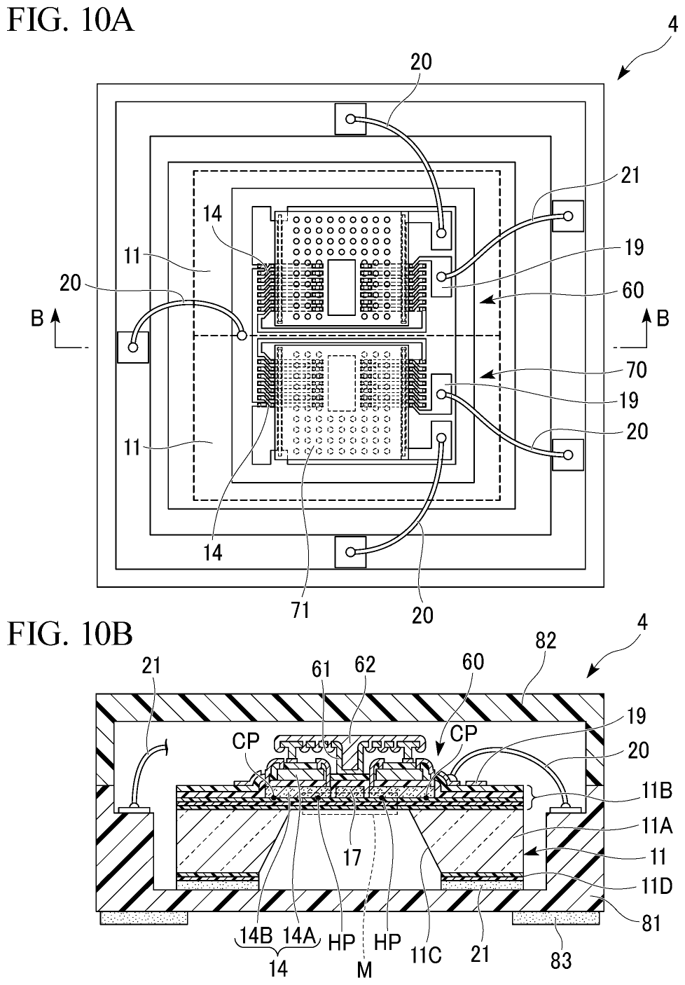

Next, a fourth embodiment will be described. FIG. 10A is a top view showing a contact combustion type gas sensor according to a fourth embodiment. FIG. 10B is a sectional view taken along line B-B of FIG. 10A. As shown in FIGS. 10A and 10B, the contact combustion type gas sensor 4 according to the present embodiment is mainly different from that of the first embodiment in that a reference gas sensor to which a reference of a catalyst is set is provided besides a gas sensor body, and in that a heater 61 also functions as a support member.

As shown in FIGS. 10A and 10B, the contact combustion type gas sensor 4 of the present embodiment includes a gas sensor body 60 and a reference device 70. The gas sensor body 60 and the reference device 70 are arranged on a base material 31, and are all covered by a filter 33.

The gas sensor body 60 includes a substrate 11, a temperature measuring element 14, and a heat transfer film 17, all of which are the same as in the contact combustion type gas sensor 1 of the first embodiment. Further, the gas sensor body 60 includes the heater 61 and a catalytic layer 62. The heater 61 functions to raise a temperature of the catalytic layer 62 and to make a temperature inside the catalytic layer 62 uniform. The heater 61 is formed of, for example, platinum (Pt). Instead of the platinum, the heater 61 may be formed of a metal such as Ti, W, Mo, Cr, Ni, Al, Cu, Ag, Au, or the like. Silicide such as TiSi, WSi, MoSi, TaSi, CrSi, NiSi, or the like, or an alloy such as NiCr, AiCu, or the like may be used for the heater 61. Further, these metals, silicides, alloys, etc. may also be used in a heater or the like in other embodiments.

Similarly to each of the above embodiment, the catalytic layer 62 is formed by applying and burning a catalytic paste produced by mixing porous alumina powder and organic vehicles (α-terpineol and di-n-butyl phthalate) on which catalytic fine particles formed of platinum (Pt) are supported. The catalytic layer 62 has an approximate T shape, and includes a leg part and a plate part having a plate shape. As shown in FIG. 10B, the heater 61 is closely bonded to a circumference of the leg part and a lower surface side of the plate part at the catalytic layer 62. For this reason, the heater 61 also includes a leg part and a plate part. The heater 61 also functions as a support of the catalytic layer 62.

The leg part of the heater 61 is provided between the temperature measuring elements 14, and stands upward to be almost perpendicular to the upper insulating layer 11B of the substrate 11. Further, an end of a bottom surface of the plate part of heater 61 is electrically connected to surface wirings 19. A driving current is supplied to the heater 61 via the surface wirings 19. Further, an outer edge at the heater 61 is covered by the catalytic layer 62.

In comparison with the gas sensor body 60, the reference device 70 includes a reference layer 71 having approximately the same shape as the catalytic layer 62 instead of the catalytic layer 62. Apart from this, the reference device 70 has almost the same constitution as the gas sensor body 60. Like the catalytic layer 62 of the gas sensor body 60, the heater 61 is closely bonded to a circumference of a leg part and a lower surface side of a plate part of the reference layer 71 in the reference device 70. The reference layer 71 is formed by applying and burning a paste produced by mixing porous alumina powder and organic vehicles (α-terpineol and di-n-butyl phthalate). In short, the reference layer 71 is different from the catalytic layer 62 in that it does not support catalytic fine particles formed of platinum (Pt).

Further, the gas sensor body 60 and the reference device 70 are attached and fixed to a base material 81 by a die bonding material 21. The base material 81 is shaped of a box whose upper side is open. A filter 82 is provided at the upper opening. The electrodes 83 are formed under the base material 81. The electrodes 83 are connected to a control device.

The contact combustion type gas sensor 4 according to the present embodiment described above operates in the same manner as the contact combustion type gas sensor 1 according to the first embodiment. Further, the contact combustion type gas sensor 4 according to the present embodiment includes the gas sensor body 60 and the reference device 70 having the reference layer 71 of the catalytic layer 62. For this reason, since a drift caused by ambient temperature, humidity, or the like can be remarkably reduced by comparing an output voltage at the reference device 70 with an output voltage at the gas sensor body 60, reproducibility in detecting hydrogen can be enhanced.

Further, the heater 61 in each of the gas sensor body 60 and the reference device 70 enters the upper insulating layer 11B in the substrate body 11A. For this reason, the heater 61 can be made to be hardly peeled from the substrate body 11A. Further, the plurality of through-holes are formed in the heater 61, and thereby the catalytic layer 62 or the reference layer 71 can be made to be hardly peeled from the heater 61.

In the gas sensor body 60 of the present embodiment, the heater 61 functions as the support. However, similarly to each of the above embodiment, the heater may be provided on the substrate, and the gas sensor body having a catalyst may be provided on the support that is thermally connected to the substrate. In the reference device of this case, the heater may be provided on the substrate, and the reference layer on which no platinum catalytic fine particles are supported may be formed on the support that is thermally connected to the heater but does not function as the heater.

### Fifth Embodiment

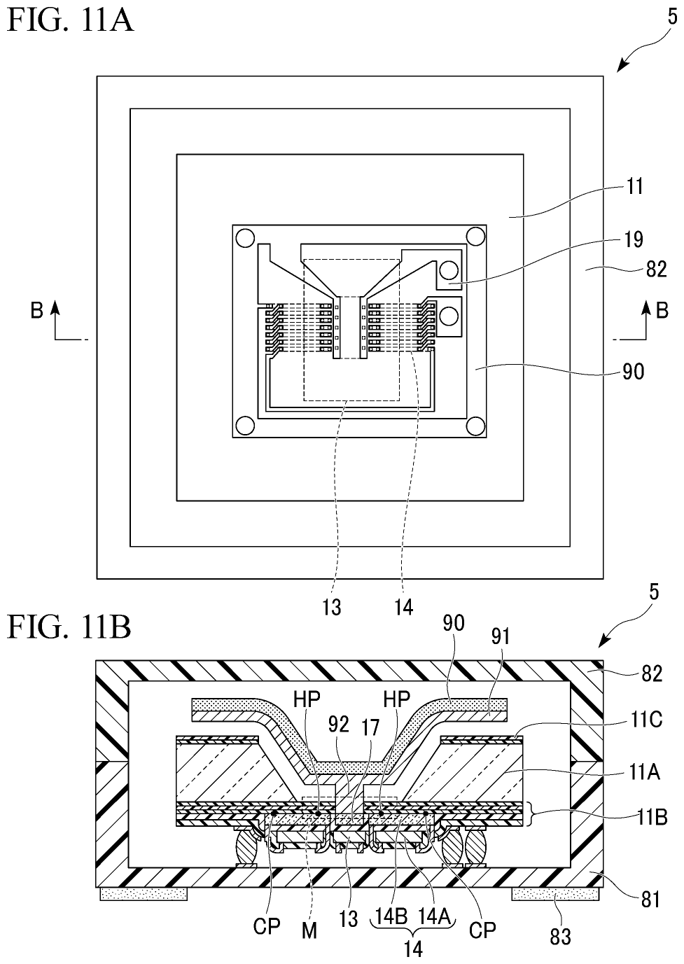

Next, a fifth embodiment will be described. FIG. 11A is a top view showing a contact combustion type gas sensor according to a fifth embodiment. FIG. 11B is a sectional view taken along line B-B of FIG. 11A. As shown in FIG. 11, the contact combustion type gas sensor 5 according to the present embodiment is mainly different from that of the first embodiment in that a catalytic layer 90 is provided on the bottom side of a substrate.

As shown in FIG. 11B, in the contact combustion type gas sensor 5, the catalytic layer 90 is provided on a side opposite to a heater 13 and a temperature measuring element 14 (on the bottom side of the substrate 11) via the substrate 11. The catalytic layer 90 is supported by a support 91. The support 91 is supported by a support leg 92. The support leg 92 stands upward in a recess 11C to be almost perpendicular to the substrate 11. Unevenness of the support 91 extends along unevenness of the bottom side of the substrate 11, and has a shape in which a portion where the recess 11C is formed protrudes. The support 91 is formed almost throughout the bottom side of the substrate 11, and has a wider area than the recess 11C when viewed in a laminating direction. A base material 81, a filter 82, and electrodes 83 have the same constitution as in the fourth embodiment except that no step is provided on the base material 81.

The contact combustion type gas sensor 5 according to the present embodiment described above operates in the same manner as the contact combustion type gas sensor 1 according to the first embodiment. Further, in the contact combustion type gas sensor 5 according to the present embodiment, the catalytic layer 90 is provided on the side opposite to the heater 13 and the temperature measuring element 14 via the substrate 11. For this reason, there is no need to provide other elements such as the heater 13 at a side at which the catalytic layer 90 is provided, and thus a space for enlarging the catalytic layer 90 can be easily secured.

While the embodiments of the present invention have been described in detail with reference to the drawings, the specific constitution is not limited to these embodiments, and a change in design or the like is included without departing the gist of the present invention.

For example, the element for measuring the temperature differential between the cold junction CP and the hot junction HP may be a temperature measuring element other than the temperature measuring element formed of a thermopile. For example, the temperature measuring element may be made up of a single thermocouple. Alternatively, an element such as a resistance temperature detector or an infrared detector may be used as the temperature measuring element. Further, in each of the above embodiments, the heater is provided, but the catalytic layer may be heated by any other means other than the heater. Alternatively, if the reaction of the detection target gas and the material in air at the catalytic layer proceeds depending on types of the catalyst and the detection target gas, and the temperature of the catalytic layer is changed, there is no need to provide the heater.

Further, for example, in the first embodiment, the through-holes 16C are provided in the support body 16B, but the through-holes 16C or the recess or the like replacing the through-holes may not be provided. Further, the area of the support body 16B when viewed in the laminating direction of the substrate may be smaller than or equal to the area of the recess 11C when viewed in the laminating direction of the substrate 11. Further, the elements of each of the above embodiments and a modification may be substituted or replaced by them of the other embodiments, or be added to the other embodiments.

What is claimed is:

1. A contact combustion type gas sensor comprising:
a substrate;
a catalytic layer that includes a catalyst for accelerating a chemical reaction of a detection target gas;
a temperature measuring element that detects at least an increase in a temperature of the catalytic layer; and
a support member that transfers heat generated by the chemical reaction toward the temperature measuring element and includes a support leg and a support body that are connected with each other, the support leg being provided between the substrate and the catalytic layer and separating the catalytic layer from the substrate, and the support body supporting the catalytic layer.

2. The contact combustion type gas sensor according to claim 1, further comprising:
a heating element that heats the catalytic layer.

3. The contact combustion type gas sensor according to claim 1, wherein the temperature measuring element includes a thermopile.

4. The contact combustion type gas sensor according to claim 1, wherein the support body includes a high thermoconductive material.

5. The contact combustion type gas sensor according to claim 1, wherein a thickness of the support body is thinner than a thickness of the catalytic layer.

6. The contact combustion type gas sensor according to claim 1, wherein at least one through-hole is provided in the support body.

7. The contact combustion type gas sensor according to claim 1, further comprising:
an insulating layer that is laminated on the substrate, the insulating layer having a membrane which is not covered by the substrate;
wherein an area of the support body when viewed in a laminating direction is larger than an area of the membrane when viewed in the laminating direction, the laminating direction being a direction in which the substrate and the insulating layer are laminated.

8. The contact combustion type gas sensor according to claim 1, wherein the catalytic layer is provided on a top of the support body and on a bottom of the support body.

9. The contact combustion type gas sensor according to claim 1, further comprising:
a thermal barrier that blocks heat discharged from the catalytic layer or the support body is provided.

10. The contact combustion type gas sensor according to claim 1, further comprising:
a reference layer that is free from the catalyst and is referred for comparison with the catalytic layer.

11. The contact combustion type gas sensor according to claim 1, wherein the support member is connected with the temperature measuring element.

12. A method for manufacturing a contact combustion type gas sensor comprising:
providing a substrate;
providing a catalytic layer that includes a catalyst for accelerating a chemical reaction of a detection target gas;
providing a temperature measuring element that detects at least an increase in a temperature of the catalytic layer; and
providing a support member that transfers heat generated by the chemical reaction toward the temperature measuring element and includes a support leg and a support body that are connected with each other, the support leg being provided between the substrate and the catalytic layer and separating the catalytic layer from the substrate, and the support body supporting the catalytic layer.

13. The method for manufacturing a contact combustion type gas sensor according to claim 12,
wherein the catalytic layer is supported on a top of the support body and a bottom of the support body, and
wherein the catalytic layer is supported on the bottom of the support body,
by forming a plurality of sacrificial films are formed on the substrate;
providing the support body along the sacrificial films, and then removing some of the sacrificial films; and
forming the catalytic layer along the remaining sacrificial films.
